(19) United States
(12) Patent Application Publication
(10) Pub. No.: US 2022/0412851 A1
(43) Pub. Date: Dec. 29, 2022

(54) Cell Collection Device II

(71) Applicant: Cytosystems Limited, Aberdeen (GB)

(72) Inventor: Sushant Dhanvijay, Aberdeen (GB)

(21) Appl. No.: 15/768,924

(22) PCT Filed: Oct. 12, 2016
(86) PCT No.: PCT/GB2016/053155

§ 371 Date: Dec. 5, 2018

(30) Foreign Application Priority Data
Oct. 19, 2015 (GB) 1518422.9
Jul. 29, 2016 (GB) 1613192.2
Oct. 11, 2016 (GB) 1617256.1

(51) Int. Cl. G01N 1/40; B01L 3/00; G01N 33/50

(52) U.S. Cl. CPC G01N 1/4077; B01L 3/502; G01N 33/5091; A61B 10/007

(57) ABSTRACT

There is provided an apparatus for collecting/isolating cells from a sample including the target cells comprising:
a membrane adapted to retain the target cells thereon,
a first port in fluid communication with the membrane provided towards a first side of the membrane,
a second port in fluid communication with the membrane provided towards a second side of the membrane,
a sample container having at least one wall defining an interior space suitable to house the sample, provided on the first side of the membrane,
a cell collection container provided towards the first side of the membrane,
a filtrate container provided towards the second side of the membrane,
a backwash fluid container provided towards the second side of the membrane, wherein the first port is movable between a first position wherein the first port is in fluid communication with the sample container, and a second position wherein the first port is in fluid communication with the cell collection container; and
   wherein the second port is movable between a first position wherein the second port is in fluid communication with the filtrate container, and a second position wherein the second port is in fluid communication with the backwash fluid container.

The present invention relates to a method and apparatus for collecting cells from a sample.

### BACKGROUND TO THE INVENTION

It is known to screen urine for the presence of abnormal cells which may indicate the presence of cancer, including cancer of the kidney, ureters, bladder or urethra. Urine samples are generally taken in hospitals. Cells are obtained from urine samples through the centrifugation of the samples. However, in busy urology departments, urine samples are often left for many hours before being centrifuged. The integrity of cells left in urine is compromised, as cells lyse or otherwise degrade under such storage conditions. The half-life of cells present in urine is dependent on the cells in question but can be as low as one hour. In addition, bacterial growth is associated with urine samples. The timing of sample processing should be adapted accordingly.

The number of prostate cells in a urine sample is extremely low, and so these must be very carefully collected. In addition, the viability of the cells must be retained. The half-life of prostate cells in urine is very low (less than one hour). The half-life of bladder epithelial cells in a urine environment is around or slightly less than 4 hours. Any degradation of the cells has a great impact on the number of cells from which may be analysed. Such degradation may be caused by lysing in the urine environment, or through adherence of cells to the filter.

The filtration of cells from biological samples such as urine is known, and is described in WO2009/087375.

US 2009/0188864 describes a microfiltration apparatus and method for separating cells whilst minimising the application of mechanical trauma to the cells and minimising membrane damage.

Known filtering devices are associated with high levels of cell adherence resulting in a reduction in the number of cells collected and recovered, and a reduction in the viability of the cells collected. Cells become lodged in the pores of known membranes and are difficult to remove, greatly reducing their viability.

The present invention provides apparatus and methods for separating cells of interest effectively and efficiently from a fluid sample whilst maintaining their viability

### SUMMARY OF THE INVENTION

According to a first aspect of the present invention, there is provided an apparatus for collecting/isolating cells from a sample including the target cells comprising:

a membrane adapted to retain the target cells thereon,

a first port in fluid communication with the membrane provided towards a first side of the membrane,

a second port in fluid communication with the membrane provided towards a second side of the membrane,

a sample container having at least one wall defining an interior space suitable to house the sample, provided towards the first side of the membrane,

a cell collection container provided towards the first side of the membrane,

a filtrate container provided towards the second side of the membrane,

a backwash fluid container provided towards the second side of the membrane,

wherein the first port is movable between a first position wherein the first port is in fluid communication with the sample container, and a second position wherein the first port is in fluid communication with the cell collection container; and

wherein the second port is movable between a first position wherein the second port is in fluid communication with the filtrate container, and a second position wherein the second port is in fluid communication with the backwash fluid container.

According to a further aspect of the present invention there is provided an apparatus for collecting/isolating cells from a sample including the target cells comprising:

a container having at least one wall defining an interior space suitable to house the sample,

a membrane adapted to retain the target cells thereon in fluid communication with the container,

at least three ports in fluid communication with the membrane, or capable of being positioned Into fluid communication with the membrane.

According to a further aspect of the present invention there is provided an apparatus for collecting/isolating cells from a sample including the target cells comprising:

a container having at least one wall defining an interior space suitable to house the sample,

a membrane adapted to retain the target cells thereon in fluid communication with the container,

means to apply a pressure differential across the membrane (generally pumping means),

characterised in that the membrane is disposed non-horizontally and the fluid flow path of the sample through the membrane is suitably non-vertical. In particular, the fluid flow path of the sample through the membrane is generally horizontal.

According to a further aspect of the present invention, there is provided a method for collecting/isolating cells from a fluid sample including the target cells comprising:

providing the apparatus as described herein

allowing the sample to begin to pass through the membrane

collecting the cells retained on the membrane.

According to a further aspect of the present invention, there is provided a method for collecting cells from a fluid sample including the target cells comprising:

placing the sample into a container in fluid communication with a membrane adapted to retain the target cells thereon,

applying an initial pressure differential across the membrane,

allowing the sample to pass through the membrane,

wherein the membrane is disposed non-horizontally, and the fluid flow path of the sample through the membrane is suitably non-vertical, in particular, the fluid flow path of the sample through the membrane is generally horizontal.

According to a further embodiment, the membrane may be disposed horizontally and the fluid flow path of the sample may be against gravity so that cells are captured on the underside of the membrane. This promotes easy dislodgement of the cells therefrom.

According a further aspect of the present invention there is provided a method of screening biological samples to identify those associated with an increased risk of a disease including:
1) collecting cells from a sample in accordance with the method as described herein;
2) removing the collected cells from the membrane;
3) measuring the presence, and/or concentration of one or more target cells in the collected cell sample of step 2);
4) analysing the measurements of step 3) for evidence of an increased risk of a disease state, in particular by comparing the measurements of step 3) with a reference score and identifying those samples with particular biomarkers/cells present and/or with increased levels of biomarkers or cells associated with an increased risk of the disease.

The membrane and filtration device of the present invention are useful in a method of screening biological samples for increased risk of particular diseases. For such embodiments, the recovery of the collected cells in a viable state is paramount. Small differences in the number of cells recovered and their viability can make a difference as to whether a sample is highlighted as being at increased risk or not. The number of cells used to make a judgment regarding the increased risk of a particular disease is very small, and thus a small difference in the number of viable cells recovered can be very important.

According to a further aspect of the present invention there is provided a method of diagnosing the risk of a patient has a disease or is likely to have the disease in the future including:

collecting cells from a sample in accordance with the method as described herein,

measuring the levels of the target cells in the sample

calculating a risk score for the patient

using the risk score to identify a likelihood that the patient has the disease or is likely to have the disease in the future.

According to a further embodiment, there is provided a system for performing the methods disclosed herein. The system can include the apparatus as disclosed herein for collecting the target cells from the patient, and generally an analytical instrument used to measure the levels of the target cells. The system also can include a suitably programmed computer for carrying out one or more steps of the methods. For example, the suitably programmed computer can carry out or assist in one or more of measuring the levels of the target cells in the sample; calculating a risk score; using the risk score to identify a likelihood that the patient suffers from a disease or has an increased likelihood of suffering from a disease in future; and displaying information related to the likelihood such as the measured target cell levels, the risk score, the likelihood that the patient suffers from a disease or has an increased likelihood of suffering from a disease in future, a reference risk score, and equivalents thereof.

According to one embodiment, the systems may also include a sample collection device adapted for obtaining a sample from a patient.

According to a further embodiment there is provided a kit of parts comprising an apparatus for collecting/isolating cells from a sample as described herein and instructions for use.

Throughout the Application, where apparatus is described as having, including, or comprising specific components, or where processes are described as having, including, or comprising specific process steps, it is contemplated that apparatus of the present teachings also consist essentially of, or consist of, the recited components, and that the processes of the present teachings also consist essentially of, or consist of, the recited process steps.

In the Application, where an element or component is said to be included in and/or selected from a list of recited elements or components, it should be understood that the element or component can be any one of the recited elements or components, or the element or component can be selected from a group consisting of two or more of the recited elements or components. Further, it should be understood that elements and/or features of a composition, an apparatus, or a method described herein can be combined in a variety of ways without departing from the spirit and scope of the present teachings, whether explicit or implicit herein.

The use of the terms “include,” “includes”, “including,” “have,” “has,” or “having” should be generally understood as open-ended and non-limiting unless specifically stated otherwise.

The use of the singular herein includes the plural (and vice versa) unless specifically stated otherwise. In addition, where the use of the term “about” is before a quantitative value, the present teachings also include the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present teachings remain operable. Moreover, two or more steps or actions may be conducted simultaneously.

As used herein, “reference” or “control” or “standard” each can refer to an amount of the target cells in a healthy individual or control population or to a risk score derived from one or more biomarkers in a healthy individual or control population. The amount of the target cells and/or the biomarker can be determined from a sample of a healthy individual, or can be determined from samples of a control population.

Features, integers, characteristics, compounds, chemical moieties or groups described in conjunction with a particular aspect, embodiment or example of the invention are to be understood to be applicable to any other aspect, embodiment or example described herein unless incompatible therewith.

### BRIEF DESCRIPTION OF THE DRAWINGS

The present invention will now be described by way of example only with reference to the accompanying figures in which:

FIGS. 1a to 1c show a representation of the method of the present invention using an apparatus of the present invention.

FIG. 2a shows an assembly including the first membrane support, the membrane (not shown) and the second membrane support (not shown).

FIG. 2b shows the first membrane support.

FIG. 2c shows the second membrane support.

FIG. 3 shows one embodiment of the apparatus according to the present invention.

FIG. 4a and FIG. 4b show cell recovery images from a sample which has undergone centrifugation.

FIGS. 5a and 5b show cell recovery images from a sample which has undergone the method of the present invention.

FIGS. 6a and 6b show a further embodiment of the apparatus of the present invention.

FIG. 7 show a further embodiment of the apparatus of the present invention.

FIG. 8 shows an embodiment of the apparatus of the present invention.

FIG. 9 shows an embodiment of the present invention.

FIG. 10 shows an embodiment of the apparatus of the present invention including a membrane provided vertically.

FIG. 11 provides a table showing the effect of including different supporting grids to the membrane in terms of cell recovery and cell damage.

FIG. 12 shows a further embodiment of the apparatus of the present invention.

FIG. 13 illustrates a void container which may be provided in gas communication with the cell collection container, and/or the filtrate container.

### DETAILED DESCRIPTION

Apparatus

There is provided an apparatus for collecting/isolating cells from a sample including the target cells comprising:

a membrane adapted to retain the target cells thereon,

a first port in fluid communication with the membrane provided towards a first side of the membrane,

a second port in fluid communication with the membrane provided towards a second side of the membrane,

a sample container having at least one wall defining an interior space suitable to house the sample, provided towards the first side of the membrane,

a cell collection container provided towards the first side of the membrane,

a filtrate container provided towards the second side of the membrane,

a backwash fluid container provided towards the second side of the membrane,

wherein the first port is movable between a first position wherein the first port is in fluid communication with the sample container, and a second position wherein the first port is in fluid communication with the cell collection container; and

wherein the second port is movable between a first position wherein the second port is in fluid communication with the filtrate container, and a second position wherein the second port is in fluid communication with the backwash fluid container.

The apparatus of the present invention generally allows a single action to change from the fluid pathway from a filtration action to a backflush action. For instance, the apparatus of the present invention generally involves the movement of a single lever to control two movements, and thus alter the apparatus from a filtering operation to a backwash operation. The apparatus of the present invention provides consistent results whoever operates it. The operator does not have to decide which stop cock/valve to alter (through turning, pinching etc.), when, and in which direction. In addition, the apparatus of the present invention generally includes less tubing than known systems,

The first and second ports may be moved between the first and second positions through, for instance, sliding or rotation of the first and second ports; generally, through rotation.

Generally, the membrane is housed within a membrane housing and movement of the membrane housing moves the first port between the first position and the second position, and moves the second port between the first position and the second position.

According to one embodiment, the membrane housing may be rotated, slid or otherwise moved to move the first and second ports between the first and second positions.

Generally during movement of the membrane housing (including rotation or sliding thereof), the membrane is not itself moved. The membrane housing is generally movable to provide access to the different containers (sample container, cell collection container, filtrate container and backwash fluid container), whilst the orientation of the membrane to the different containers remains constant.

According to one embodiment, the first port may include a first channel leading towards the sample container and/or the cell collection container, and the second port may include a second channel leading towards the filtrate container and/or the backwash fluid container. The channels may be moveable between the first and second positions, and the membrane housing may maintain a static position.

The first port may comprise or consist of an aperture in the membrane housing, and the second port may comprise or consist of an aperture in the membrane housing wherein the first and/or second port may include a channel leading from the inside wall of the membrane housing towards the membrane. In such embodiments the sample container, the cell collection container, the filtrate container and the backwash fluid container are provided externally of the membrane housing.

Generally, the first port is in the first position when the second port is in the first position and a fluid pathway is provided from the sample container, through the membrane, to the filtrate container.

Suitably, when the first port is in the second position, the second port is in the second position and a fluid pathway is provided from the backwash fluid container, through the membrane, to the cell collection container.

Generally, the first port provides fluid communication to either the sample container or the cell collection container, and the second port provides fluid communication to either the filtrate container or the backwash fluid container. The membrane housing typically precludes fluid access between the membrane and the cell collection container where the first port is in the first position, and the membrane housing typically precludes fluid access between the membrane and the sample container where the first port is in the second position. Likewise, the membrane housing typically precludes fluid access between the membrane and the backwash fluid container where the second port is in the first position, and the membrane housing typically precludes fluid access between the membrane and the filtrate container where the second port is in the second position.

According to one embodiment, the first port is movable between the first position, the second position and a closed position, and in the closed position, movement of fluid from the sample container to the membrane is precluded and movement from the membrane to the cell collection container is precluded. The second port may be movable between the first position, the second position and a closed position, and in the closed position movement of fluid from the membrane to the filtrate container is precluded and movement from the backwash fluid container to the membrane is precluded. Where the first and second ports are provided in the closed position, a pressure differential may be applied across the membrane.

Where the first and second ports are in the closed position, a vacuum or positive pressure may be generated within the membrane housing. This potential energy may be retained within the membrane housing until the ports are moved from the closed position to the first position, whereby the sample is forced through the membrane. The ports may then be moved to the closed position to allow a pressure differential to be applied across the membrane. The ports are then moved from the closed position to the second position and the backwash fluid is forced through the membrane to the cell collection container.

The ports are generally adapted to be non-removable, in particular during the use of the apparatus in a filtration process.

The apparatus may comprise more than two ports in fluid communication with the membrane, for example allowing more than one back flushing fluid to be used.

According to one embodiment, the apparatus may include a flow restriction orifice between the second port and the filtrate container. The narrowest width or diameter of the first port is generally at least two times greater than the narrowest width or diameter of the flow restriction orifice, typically at least four times greater. The flow of fluid from the sample container to the membrane is generally greater than the flow fluid from the membrane to the filtrate container. This minimises the force applied to cells within the sample, and reduces the risk of cell damage.

The flow restriction orifice is generally located between the second port and the filtrate container, meaning that the flow of backwash fluid to the membrane is not restricted by the flow restriction orifice.

According to one embodiment, the narrowest diameter of the first port is around 1 mm or more, generally 1 to 3 mm, typically 1 to 2 mm. The narrowest diameter of the flow restriction orifice is generally more than 0.45 mm, typically around 0.5 to 0.6 mm.

According to one embodiment, the sample container may include a mechanism to automatically block the passage between the sample container and the membrane once the sample container has emptied of the sample. Such a mechanism prevents air from being sucked from the sample container to the membrane by blocking the exit from the sample container to the membrane. The inclusion of such a mechanism reduces the risk of cell damage by minimizing force on the cells. The cells are not forced onto or through the membrane through the flow of air which is sucked through from the sample container. This maximises cell collection rates and the viability of collected cells. In addition, the membrane will be kept wet, as air flow will be minimised or prevented. Drying of the membrane increases the risk of damage to cells compared to cells that are maintained in solution.

The mechanism to automatically block the passage between the sample container and the membrane once the sample container has emptied of the sample generally comprises a valve, typically a floating valve, for instance in the form of a body which floats in the sample and blocks the passage between the sample container and the membrane once the sample container has been emptied. According to one embodiment, the mechanism comprises a floating ball shut-off valve. When in the closed position, this mechanism may remove the pressure differential across the membrane, and thus the sample may stop passing through the membrane when the mechanism is in the closed position. Although the mechanism may move to the closed position once the sample container has been emptied, a proportion of the sample may not yet have passed thorough the membrane.

When the mechanism is in the closed position, the pressure differential across the membrane is generally reduced by at least 90%, suitably the pressure differential is removed.

According to one embodiment, a fluid proportion of the sample is retained on the first side of the membrane upon removal of the pressure differential across the membrane, suitably upon closure of the mechanism to block the passage between the sample container and the membrane. Generally, 5 to 20 vol. % of the sample is provided on the first side of the membrane upon removal of the pressure differential, suitably around 10 vol. %.

The first side of the membrane generally faces around 180° from the second side of the membrane.

Each of the containers has at least one wall defining an interior space suitable Lu house a liquid.

Generally, the backwash fluid is a preservative for human cells, generally comprising alcohol. The skilled man will be well aware of suitable fluids.

According to a further aspect of the present invention there is provided an apparatus for collecting/isolating cells from a sample including the target cells comprising:

a container having at least one wall defining an interior space suitable to house the sample,

a membrane adapted to retain the target cells thereon in fluid communication with the container,

at least three ports in fluid communication with the membrane.

The provision of at least three ports in fluid communication with the membrane provides significant advantages over standard filtration apparatus which include only two ports. In particular the membrane does not have to be repositioned during the filtration and back flush processes. The multi-port system of the present invention allows fluids to pass through the membrane in both directions without substituting the containers (for instance, substituting the filtrate collection housing with the container housing the backflush fluid). This simplifies a filtration process involving the apparatus, and reduces the risk of spillage or inadvertent loss of sample. The inclusion of at least three ports prevents mixing between the filtrate and the backflush fluid which may cause further clogging of the membrane.

Suitably the membrane is housed within a membrane housing and the apparatus includes three distinct ports into the membrane housing. The three or more ports provide at least three distinct fluid pathways to the membrane, generally at least three distinct fluid pathways in to the membrane housing.

Generally, one of the ports allows fluid communication between the membrane and the sample container, one of the ports allows fluid communication between the membrane and a filtrate collection housing and one of the ports allows fluid communication between the membrane and a container housing backflush fluid (suitably one or more of cell preserving fluid, washing fluid and buffer).

At least one of the ports is in fluid communication with a first side of the membrane, facing towards the sample container and at least one of the ports (generally at least two of the ports) is in fluid communication with a second side of the membrane facing away from the sample container.

At least one of the ports provides a fluid pathway between the sample container and a first side of the membrane and generally at least one of the ports provides a fluid pathway from the second side of the membrane to a filtrate collection housing. Typically, at least one of the ports provides a fluid pathway between a container housing the backflush fluid and the second side of the membrane.

Generally, the ports are designed or adapted to be non-removable, in particular during a filtration process. However, the ports may be independently moveable between an open position where fluid communication to the membrane is possible, and a closed position where fluid communication to the membrane is not possible.

According to one embodiment, the apparatus includes at least four ports in fluid communication with the membrane.

Generally, at least two of the ports are in fluid communication with a first side of the membrane, facing towards the sample container and at least two of the ports are in fluid communication with a second side of the membrane facing away from the sample container.

Typically, where the apparatus comprises at least four ports, one of the ports provides a fluid pathway from the first side of the membrane to a container suitable for collecting the cell sample and backwash fluid.

Generally, the ports are capable of providing access to the membrane throughout any filtration process.

Suitably the apparatus described herein is used to perform the method as disclosed herein.

According to a further aspect of the present invention there is provided an apparatus for collecting/isolating cells from a sample including the target cells comprising:

a container having at least one wall defining an interior space suitable to house the sample,

a membrane adapted to retain the target cells thereon in fluid communication with the container,

means to apply a pressure differential across the membrane (generally pumping means),

characterised in that the membrane is disposed non-horizontally and the fluid flow path of the sample through the membrane is suitably non-vertical. In particular, the fluid flow path of the sample through the membrane is generally horizontal.

Generally, the apparatus comprises at least three ports in fluid communication with the membrane.

According to one embodiment, the membrane is disposed at an angle of 30 to 100° relative to the fluid flow path of the sample through the membrane, generally wherein the membrane is disposed substantially diagonally (around 45°), or substantially perpendicularly (around 90°) to the fluid flow path of the sample through the membrane.

The apparatus disclosed herein is generally suitable for use in the method(s) disclosed herein, and/or is used to perform the method(s) disclosed herein.

The apparatus generally includes control means to reduce or remove the pressure differential across the membrane. According to one embodiment, the apparatus includes control means which remove the pressure differential before all of the sample has passed through the membrane. The control means may comprise a valve (for instance a floating valve) which move to remove the pressure differential across the membrane before the sample has depleted. For instance, the control means may move to block access from the membrane to the vacuum, or pump which is establishing the pressure differential.

The means to apply a pressure differential are generally capable of applying a pressure differential across the membrane in opposite directions.

The apparatus generally also includes a filtrate collection housing, suitable for collection of the filtrate therein.

The time period for which the sample is maintained on the membrane is generally maximised in order to ensure as high a rate of cell collection as possible. The apparatus generally includes an inlet providing fluid communication between the sample container and the membrane, and an outlet providing fluid communication between the membrane and the filtrate collection housing. In order to achieve this, the size of the inlet channel from the sample container to the membrane may be larger than the size of the outlet channel from the membrane to a housing used to collect the filtrate. In particular, the outlet channel from the membrane to the filtrate collection housing may include a flow restriction orifice, narrowing the outlet channel.

The dimensions of the inlet channel from the sample container to the membrane, the outlet channel from the membrane to a housing used to collect the filtrate and the flow restriction orifice depend on the proposed use of the apparatus, including the type of sample, the type of cells and the volumes to be filtered.

According to one embodiment, flow restriction orifice has a maximum width or diameter of 0.1 to 2 mm, generally 0.1 to 1 mm, typically 0.3 to 0.7 mm, more suitably around 0.5 mm. A flow restriction orifice having a minimum width or diameter of around 0.5 mm is associated with certain advantages where the sample has a volume of 30 to 100 ml. In particular, a flow restriction orifice having such dimensions controls the flow rate well, and maximises contact between the sample and the membrane.

According to one embodiment, the inlet channel from the sample container to the membrane has a minimum width or diameter of 1 to 10 mm, generally 1 to 7 mm, suitably around 1 to 2 mm.

The side of the membrane facing away from the sample container (and generally facing towards the filtrate collection housing) is generally supported by a first membrane support. Generally, the first membrane support is in the form of a grid, or mesh and should include support for the membrane at least every 5 mm². Typically, the first membrane support is in the form of a mesh having a mesh size of 1 to 2 mm. Generally, the pore size is 0.5 to 2 mm, typically around 1 mm and there is a space of 0.5 to 2 mm, typically around 1 mm between each pore.

According to one embodiment, the membrane is supported on both sides with a membrane support.

Generally, the grid or mesh size of the second membrane support is as big as possible to allow as much membrane area as possible to maximise contact between the sample and the membrane in order to maximise the cell collection rate during back flushing.

The side of the membrane facing towards the sample container (and generally facing away from the filtrate collection housing) is generally supported by a second membrane support. This is useful for support during a back flush step to collect the target cells from the membrane. Generally, the second membrane support supports the membrane at greater unit areas than the first membrane support. Generally, the second membrane support is in the form of a grid, and generally provides support for the membrane at least every 2 cm². Typically, the first membrane support is in the form of a grid, wherein the spacing between adjacent supports on the grid is 10 to 20 mm, typically around 14 to 15 mm.

According to one embodiment, the grid or mesh size of the first membrane support (supporting the membrane during filtering) is at least 5 times less than the grid or mesh size of the second membrane support (supporting the membrane during back flushing).

There is suitably a gas release outlet in the sample container to release any air or other gases from the sample container. This reduces any pressure build up in the sample container and reduces any turbulence of the sample passing through the membrane. Generally, the gas release outlet is a valve.

The apparatus is generally sealed, in particular the portion of the apparatus from the sample container to the membrane, and past the membrane towards the filtrate collection housing is sealed so that no fluid (gas and liquid) can escape. This allows a complete or partial vacuum to be established past the membrane towards the filtrate collection housing to suck the sample through the membrane. The skilled man would be well aware of several methods of generating a full or partial vacuum. Mention may be made of methods including the use of syringes and vacutainers.

In addition, the sealing of this portion of the apparatus allows the possibility of the establishment of a complete or partial vacuum between the sample container and the membrane which can prove useful in any back flushing step, allowing cell preservative fluid to be sucked back through the membrane to collect cells from the membrane collected during the filtration process.

The apparatus of the present invention generally includes a container including one or more of a cell preserving fluid, washing fluid or buffer. This container is generally in controllable fluid communication with the membrane, where the fluid communication can be blocked, or controlled as required. This container is generally in fluid communication with the surface of the membrane facing towards the filtrate collection housing. This container is useful during the optional back flush step referred to above where the cell preserving fluid, washing fluid or buffer may pass or be pumped through the membrane to collect the cell containing residue.

According to one embodiment, filtrate collection housing and the container including one or more of a cell preserving fluid, washing fluid or buffer are both connected to the membrane, but the membrane is in fluid communication with only one of these containers at any one time.

The membrane may be formed from any suitable material and particular mention may be made of nylon, polycarbonate and polyester membranes.

Generally, the pores extend through the depth of the membrane providing a passage or channel through which the sample passes.

The pores of the membrane generally extend from the surface of the membrane facing towards the sample container to the surface of the membrane facing away from the sample container and generally facing towards the filtrate collection housing.

The diameter of the pores is suitably smaller than the diameter of the target cells to be collected. The target cells cannot pass into or through the pores easily, and are retained by the membrane. It will be understood that the average diameter of the pores is dependent on the cells to be collected. Generally, the pores have an average diameter of less than 50 μm; suitably 0.1 to 20 μm; typically, 1 to 10 μm; preferably 4 to 10 μm; advantageously 4 to 9 μm.

According to one embodiment, 90% of the pores of the membrane have a diameter of 0.1 to 20 μm.

According to one embodiment, the average pore size is 3 to 10 μm, typically 3 to 8 μm, suitably 5 to 8 μm. Suitably 90 to 99% of the pores fall within this pore size range.

Surprisingly, pre-malignant cells having diameters far greater than the diameter of a pore, may still pass through the pore due to the cells extruding. The present inventors have noted cells having a diameter of 8 to 10 μm extruding themselves through pores having a diameter of 1 to 2 μm. Although the average pore size of the membrane of the present invention is typically 4 to 10 μm, the non-vertical fluid flow path and the control of the pressure differential across the membrane largely precludes the target cells being extruded through the pores of the membrane.

According to one embodiment, the membrane is nylon and the average pore size is around 5 μm.

According to a further embodiment, the membrane is polycarbonate (for instance those sold under trade name Cyclopore®) or polyester and the average pore size is around 8 μm.

Suitably the distribution of pores on the sample contacting surface of the membrane is relatively uniform and the pores are fairly evenly spaced. Generally, the membrane comprises 100,000 to 300,000 pores per unit mm², typically 150,000 to 200,000 pores per unit mm². The average distance between the pores may be 20 to 50 microns, typically 20 to 40 microns, suitably 25 to 35 microns, more suitably around 30 microns.

The membrane of the present invention may comprise pores in more than one plane.

The size of the membrane is dependent on the apparatus with which it may be used, and the fluid sample to be filtered. The membrane may suitably be up to 10 cm², typically up to 5 cm², generally 3 to 5 cm², generally 0.5 to 2 cm².

It will be appreciated that the shape of the membrane is determined by the filtration device for which it is adapted for use. The membrane may be formed into any suitable shape. Particular mention may be made of substantially flat shapes including substantially circular, square, rectangular and oval filtration patches. Mention may also be made of 3d shapes such as conical and tubular forms. For any shape, filtration may be provided by pores in more than one plane.

The dimensions of the membrane including length, width and thickness are dependent on the proposed use of the apparatus, including the type of sample, volume of sample and target cells of interest.

The membrane is suitably 10 to 15 μm thick.

According to one embodiment, the cell collection container and/or the filtrate container optionally comprise a compartment termed a “void container”, allowing gas access from the void container to the cell collection chamber, or alternatively from the void chamber to the filtrate container. This acts to reduce the negative pressure/allows creation of a partial vacuum. Optionally, there is a one-way valve which serves to allow air out of the void chamber and seals again when the pressure is equalised which occurs after the preservative (or filtrate) is collected within the collection containers, this serves to prevent preservative (or filtrate) from leaking into the void chamber.

The volume of the void chamber may be modified to increase or reduce the partial vacuum. Typically, the void volume is between 8 ml and 12 ml in the preservative cell collection section and 20-40 ml in the filtrate collection container.

As used herein, “sample” refers to any biological sample taken from an animal or human (generally a human), including blood, blood plasma, blood scrum, cerebrospinal fluid, bile acid, saliva, synovial fluid, pleural fluid, pericardial fluid, peritoneal fluid, sweat, faeces, nasal fluid, ocular fluid, intracellular fluid, intercellular fluid, lymph urine, tissue, liver cells, epithelial cells, endothelial cells, kidney cells, prostate cells, blood cells, lung cells, brain cells, adipose cells, tumour cells, mammary cells, cavity washes (including lung, colorectal or nasal cavity washes) and semen.

Generally, the sample consists of, essentially consists of or comprises a fluid selected from the group consisting of blood, saliva, cavity washes (suitably lung, mouth, colorectal or nasal cavity washes), semen, urine or a suspension comprising a biomass such as stools. Typically, the sample is urine.

The target cells may be eukaryotic cells, in particular cells indicative of cancer, such as cancer of the renal system, pelvis, prostate or hyper nephroma. Particular mention may be made of bladder cells, in particular bladder epithelial cells, prostate cells, circulating tumour cells (CTC), stem cells.

Method of Collection

According to a further aspect of the present invention, there is provided a method for collecting/isolating cells from a fluid sample including the target cells comprising:

providing the apparatus as described herein

allowing the sample to begin to pass through the membrane

collecting the cells retained on the membrane.

The pressure imparted on the sample is generally standardised and any user variability is therefore reduced or removed.

Generally, the flow of the sample through the membrane is stopped before all of the sample has passed through the membrane, thus ensuring that a fluid portion of the sample is retained on the membrane.

Typically, an initial pressure differential is applied across the membrane, and this causes the sample to begin to pass through the membrane. Alternatively, the membrane itself may be pulled or pushed through a fluid sample to cause the sample to pass through the membrane.

According to one embodiment, the system is pre-flushed with a liquid prior to the filtration process in order to reduce or eliminate any peak in pressure differential across the membrane where air is sucked through the membrane, prior to the sample passing through.

Generally, the application of a vacuum is used to create a pressure differential. However, according to one embodiment, magnets may be used to create a pressure differential.

The flow of the sample through the membrane is generally stopped before all of the sample has passed through the membrane thus ensuring that a fluid portion of the sample is retained on the membrane. The retention of a fluid portion of the sample on the side of the membrane towards the container ensures a reduction of forces on the cells retained on the membrane and thus decreases the risk of cell damage. There is therefore a decreased risk of the morphology of the cells changing. Cells captured during the filtration process of the present invention are more likely to retain a normal morphology and normal characteristics. This is very important when the captured cell samples are screened as differentiation of cancer cells from normal cells is partly made on the basis of morphology, and texture.

Generally, the initial pressure differential is reduced by at least 90% (generally by around 100%) before all of the sample has passed through the membrane, thus ensuring that a fluid portion of the sample is retained on the membrane.

Typically, the flow of the sample through the membrane is stopped by removing the pressure differential across the membrane to ensure that at least 1 vol. % of the sample is retained on the side of the membrane towards the container, generally 1 to 20 vol. %, suitably around 10 vol. %. Typically, the initial volume of the sample is around 100 ml or less.

This prevents the membrane drying out, and allows or ensures that the target cells remain in a liquid medium during and immediately following the filtration process. This reduces the risk of the cells adhering to the membrane and reduces the risk of cellular damage, thus promoting a high collection rate of non-damaged target cells with their integrity preserved.

The aim of the methods of the present invention is not merely to separate the target cells from the sample, but to isolate them in a form from which they may be collected and analysed. The rate of collection of the target cells from the sample must be high as the target cells are generally present in samples at very low concentrations. In addition, the morphology of the cells must be retained to allow accurate analysis thereof. The method of the present invention reduces the risk of damaging the cells of interest and maximises the rates of collection. The cells which are generally of interest have a morphology which is easily disrupted. If too much pressure is applied to the cells, their morphology can become disrupted and they can be forced through membrane pores which are smaller than the diameter of the cells. The cells can thus be pulled into the filtrate, reducing the percentage collection rate. Alternatively, or additionally the cells can become lodged within the pores, or adhered to the surface of the membrane which additionally reduces the percentage collection rate, and increases the risk of cells becoming damaged.

The membrane is typically disposed non-horizontally, and the fluid flow path of the sample through the membrane is suitably non-vertical. In particular, the fluid flow path of the sample through the membrane is generally horizontal.

The preferred, non-vertical fluid flow of the sample through the membrane is believed to maximise the cell collection rate. After colliding with the membrane surface, the cells may be pulled, or fall away from the membrane surface under the action of gravity. This reduces the pressure pulling the cells into the pores of the membrane associated with a non-vertical fluid flow compared to that associated with a vertical fluid flow. A vertical fluid flow can be associated with high membrane adhesion and low cell collection rates as a relatively high number of cells are pulled through the membrane into the filtrate due to gravitational forces applied to the filtration process. There is less adhesion of cells on the surface of the membrane, and less risk of cells being lodged in the pores of the membrane, or being pulled through the membrane into the filtrate.

Accordingly, where the membrane of the present invention is disposed non-horizontally the cell collection rate is maximised and the rate of cellular damage of the target cells is decreased. The membrane may disposed within 30 to 100° from the direction of the sample fluid flow path, suitably substantially diagonally (around 45°) or substantially perpendicularly (around 90°) compared to the sample fluid flow path. According to one embodiment, the membrane is disposed substantially vertically.

According to one embodiment, the membrane is disposed substantially horizontally and the fluid flow path of the sample through the membrane is substantially vertical.

The fluid flow may be against gravity, with the cells captured on the underside of the membrane. Such an embodiment is also associated with relatively low membrane adhesion, and high cell collection rates.

According to a further aspect of the present invention, there is provided a method for collecting/isolating cells from a fluid sample including the target cells comprising:

placing the sample into a container/housing in fluid communication with a membrane adapted to retain the target cells thereon,

applying an initial pressure differential across the membrane,

allowing the sample to pass through the membrane,

wherein the membrane is disposed non-horizontally, and the fluid flow path of the sample through the membrane is suitably non-vertical, in particular, the fluid flow path of the sample through the membrane is generally horizontal.

The membrane may be disposed at an angle of at least 20° relative to the fluid flow path of the sample through the membrane,

According to one embodiment, the fluid flow path of the sample through the membrane is substantially horizontal, and the membrane is disposed diagonally relative to the fluid flow path. Alternatively, the membrane may be disposed substantially perpendicularly relative to the fluid flow path, generally the membrane is disposed substantially vertically.

The initial pressure differential may be applied by applying a vacuum or partial vacuum to the side of the membrane facing towards the filtrate collection housing and generally facing away from the container. Typically, the initial pressure differential is applied through the establishment of a partial vacuum.

Accordingly, the sample is suitably sucked through the membrane through the establishment of a partial or complete vacuum. This is in contrast with many standard filtration processes which involve the establishment of a pressure the sample feed side of the membrane to push the sample through the membrane.

Alternatively, the initial pressure differential may be applied through the application of a positive pressure above the sample, to push the sample through the membrane.

Following collection of the target cells from the sample, the residue may be mixed with a fluid such as one or more of a cell preserving fluid, washing fluid and/or buffer. Following mixing of the residue with a cell preserving fluid, the resultant mixture may undergo prolonged periods of storage without a significant decline in the number of target cells. Assessment for the presence and/or concentration of particular cells and optionally biomarkers of interest in the sample may be delayed for several days or weeks which can be advantageous in busy hospital departments. If the mixture is stored under refrigeration, storage periods of 25 to 27 days are possible.

Generally, the method of the present invention involves the back flushing of the membrane with a fixative fluid or a cell preservative fluid to collect the cells.

The back flushing step involves establishing a secondary pressure differential across the membrane opposite to the initial pressure differential and allowing a cell preservative fluid to pass through the membrane.

The secondary pressure differential generally involves the establishment of a lower pressure towards the side of the membrane facing towards the container.

The secondary pressure may be of the same magnitude as the initial pressure, or of a different magnitude. Generally, the secondary pressure is greater in magnitude than the initial pressure. Typically, the secondary pressure differential is applied through the establishment of a vacuum (generally a complete vacuum as opposed to a partial vacuum). Following collection of the target cells from the membrane (typically through the back flushing process described above), the sample may be directly analysed, or may be dispatched to a laboratory for further processing.

According to one embodiment of the present invention, the flow rate of the sample through the membrane is optimised primarily through controlling the pressure differential across the membrane, but also through limiting the size of the outlet from the membrane to the filtrate collection housing relative to the sample container The time period for which the sample is retained on the membrane is generally maximised in order to ensure as high a rate of cell collection as possible. According to one embodiment, the sample is 30 to 100 ml and the filtration process (steps a. to e.) generally take 0.5 to 5 minutes, typically 1 to 3 minutes, suitably 1 to 2 minutes.

Generally, the cells are separated or collected from the sample as soon as possible after production of the sample (generally removal of the sample from a human or animal body). Cells degrade, particularly through lysing when left in fluids such as urine for prolonged periods, and the sooner they are separated from such fluids, the more will be detected in the sample. The rate of degradation of the cells is dependent on both the type of cell and the type of fluid. Generally, the method of separation should be conducted less than one day after production of the sample, suitably less than six hours, more suitably less than two hours; preferably less than 30 minutes after production of the sample.

Prostate cells are estimated to have a half-life of less than one hour in urine, and where the method of the present invention is used to separate prostate cells from a urine sample, the method is preferably conducted 30 minutes or less from production of the sample. Bladder epithelial cells are believed to have a half-life of around four hours in urine, and where the method of the present invention is used to separate bladder epithelial cells from a urine sample, the method is preferably conducted 3 hours or less from production of the sample.

According to one embodiment, the method of the present invention collects approximately the same number of cells as centrifugation methods; suitably at least 90% of the number of cells/biomarkers; more suitably at least 95%; preferably at least 99%; advantageously 100 to 110% of the number of cells collected through centrifugation methods.

In addition, the viability of the cells collected according to the method of the present invention is increased compared to known methods of filtering samples to recover cells.

According to one embodiment of the present invention, the filtrate collected following the filtration process of steps a. to e. may be repeated by collecting the filtrate and placing it into the sample container to pass through the membrane again.

Samples may be obtained from mammals other than humans, (e.g. for testing purposes or veterinary therapeutic purposes), and if so the methods are carried out under substantially the same conditions as described above. A patient, as used herein, is generally a mammal, preferably a human, and including a non-human primate, cow, horse, pig, sheep, camel, goat, dog, cat or rodent.

Method of Screening or Diagnosis

According a further aspect of the present invention there is provided a method of screening biological samples to identify those associated with an increased risk of a disease including:
1. collecting cells from a sample in accordance with the method as described herein,
2. removing the collected cells from the membrane;
3. measuring the presence, and/or concentration of one or more target cells in the collected cell sample of step 2;
4. analysing the measurements of step 3 for evidence of an increased risk of a disease state, in particular by comparing the measurements of step 3 with a reference score and identifying those samples with particular biomarkers/cells present and/or with increased levels of biomarkers or cells associated with an increased risk of the disease.

In order to identify an increased risk of a disease, the cells or biomarkers of interest should be viable. Damage to the target cells may be associated with their removal from a membrane following adherence thereto. Cells may also enter pores of a membrane, before getting lodged in the lumen of the membrane, and this is associated with a decrease in the viability of cells. The method and apparatus of the present invention is associated with a lower rate of cell adherence than known filtration methods and apparatus. There is therefore a decreased associated risk of cell damage, and an increased viability rate of the collected cells. The method of the present invention, thus provides a more reliable, accurate method of screening for increased risk of a particular disease.

The apparatus of the present invention generally provides a single device for collecting cells, concentrating cells and preserving cells.

The apparatus of the present invention generally includes a detachable membrane, and optionally a detachable membrane housing. The membrane (and optionally the membrane housing) may be disposed, and the apparatus may be reassembled with a replacement membrane (optionally with housing).

According to a further aspect of the present invention there is provided a method of diagnosing the risk of a patient having a disease or is likely to have the disease in the future including:

collecting cells from a sample in accordance with the method as described herein,

measuring the levels of the target cells in the sample

calculating a risk score for the patient

using the risk score to identify a likelihood that the patient has the disease or is likely to have the disease in the future.

The presence and/or concentration of the target cells, optionally as well as one or more biomarkers or cells may be determined using any suitable method known to one skilled in the art. Particular mention may be made of immunoassay, colorimetric assay, turbidometric assay and flow cytometry. The sample may be contacted with a label that binds to the target cells to provide emission of a labelling signal. Suitable labels include radioactive isotopes, fluorophores, enzymes, dyes, ligands and nanoparticles.

The collected cell sample may be contacted with a biomarker known to bind with a particular cell, and the presence and concentration of the biomarker may be assessed.

The method may include the step of measuring the presence and/or concentration of more than one cell to provide a cell concentration profile for comparison in step 4. Each of these measurements may be measured using the same or different assays. Generally different target molecules are detected using different assays.

Cell counts are generally assessed using a haemocytometer.

A risk score may be calculated on the basis of other factors as well as the levels of the target cells present in the sample, for instance levels of other biomarkers indicative of the presence or likelihood of the disease. The level of other biomarkers may be assessed from the sample or from other samples taken from the patient.

A risk score may be calculated by weighting measured levels of target cells and optionally other biomarkers.

The risk score can be compared to a reference risk score (or standard risk score). A reference risk score can be a standard or a threshold. The threshold can be a lower threshold, an upper threshold, or a threshold having an upper limit and a lower limit.

A threshold risk score may be calculated with reference to average measurements of the presence and concentration of the particular cell(s) in samples taken from individuals having the disease. The method may include calculating a threshold risk score by weighting the concentration of more than one cell in a particular sample.

Alternatively, the reference score may be the average score of the presence and/or concentration of a particular cell.

The methods of the present invention may be used to identify the likelihood that the patient is likely to have the disease in the future. Generally, in the future may be interpreted to mean within the 6 years immediately following the time at which the sample was taken from the patient, generally within 2 years, suitably within 1 year, more suitably within 3 to 6 months of the time at which the sample was taken from the patient.

The method of the present invention does not generally provide a definitive diagnosis of a disease but generally highlights samples having an associated increased risk of the disease for further tests and/or consideration by trained personnel. If a patient is found to have an increased risk of the disease, he/she may be offered increased monitoring.

Suitable diseases for screening include precancerous or cancerous conditions including those of the skin, oral cavity, larynx, lung, bladder, vulva, breast, digestive tract, renal system, pelvis, prostate, hyper nephroma or any other organ. The method of the present invention may be used in the identification of samples at high risk of basal cell carcinomas, squamous cell carcinomas, including those of the head and neck, bladder tumours.

In various embodiments of the present teachings, the methods of diagnosing a disease or increased likelihood of a disease may include inputting into a computer including a computer readable medium measurements of the level of the target cells, optionally inputting into the computer the level of one or more additional biomarkers; and causing the computer to calculate a risk score for the patient by weighting the measured levels of biomarkers, thereby determining the risk of the disease or the risk of the patient developing the disease.

Kit

According to an aspect of the present invention, there is provided a kit of parts comprising an apparatus for collecting/isolating cells from a sample as described herein and instructions for use.

The kit may include reagents and materials for measuring the levels of the target cells in the sample, for example reagents appropriate for detecting cells using, for example, flow cytometry.

Kits can also include a control, which can be a control sample, a reference sample, an internal standard, or previously generated empirical data. The control may correspond to a normal, healthy individual or an individual having a known disease status. In addition, a control may be provided for the target cells or the control may be a reference risk score.

Kits can further include instructions for performing the methods described herein and/or interpreting the results, in accordance with any regulatory requirements. In addition, software can be included in the kit for analysing the detected target cell levels, calculating a risk score, and/or determining the likelihood of the patient having, or having an increased risk of developing a disease. Preferably, the kits are packaged in a container suitable for commercial distribution, sale, and/or use, containing the appropriate labels, for example, labels including the identification of the target cells.

### DETAILED DESCRIPTION OF EXEMPLARY EMBODIMENTS

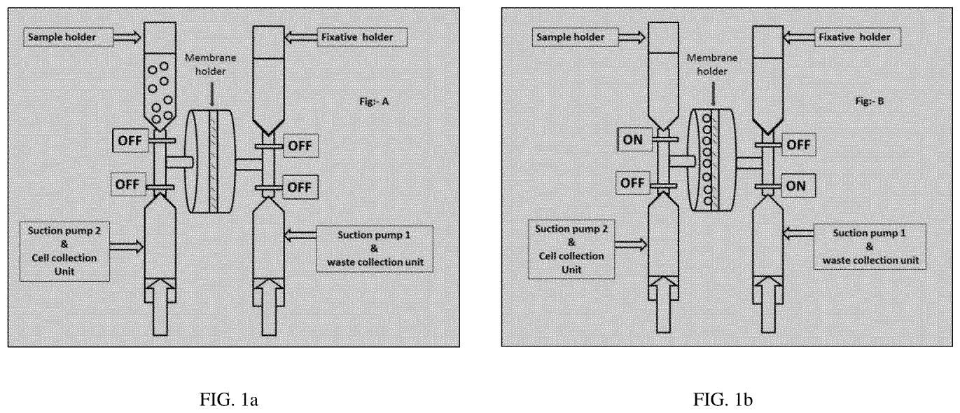

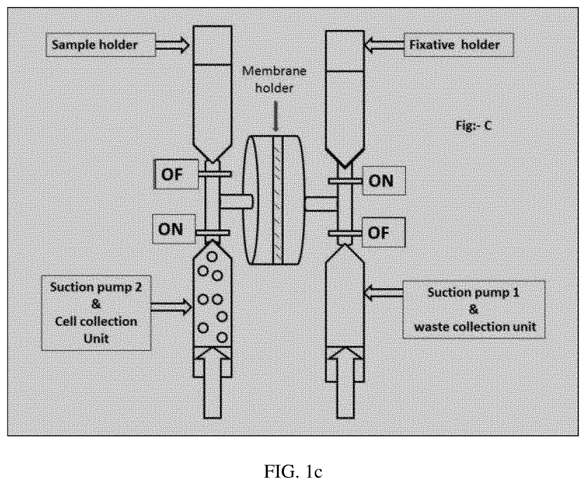

FIGS. 1a to 1c provide schematic representations of an embodiment of the apparatus 1 in a cell collection method. The sample container 2 is provided vertically. A pressure differential is applied across the membrane 5 (provided within a membrane holder 6) caused by the application of a partial vacuum due to suction pump one 7. The sample passes through the membrane 5, into the filtrate container 8. A second pressure differential is then applied across the membrane 5 in the opposite direction and the fixative fluid passes from a backwash fluid container 9 through the membrane 5, to the cell collection container, 10, suspending the cell residue collected on the left hand surface of the membrane (as shown).

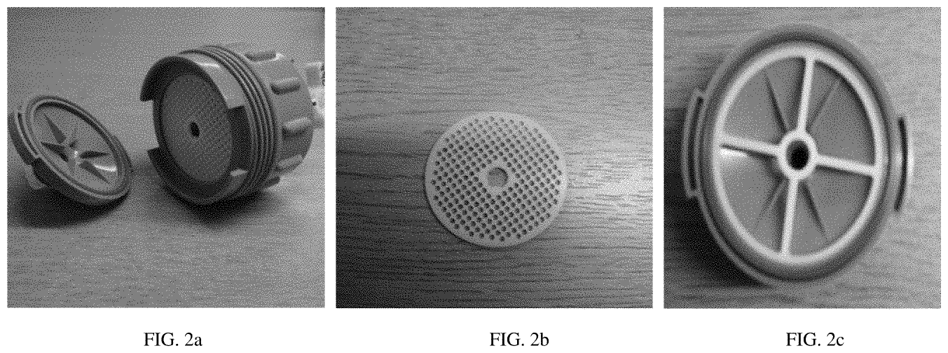

FIG. 2a shows an assembly including the first membrane support, the membrane (not shown) and the second membrane support (not shown). The first membrane support is shown in detail in FIG. 2b. This support is intended in use to support the membrane from the filtrate collection container facing side, and this is shown as the right hand side of the membrane provided in FIG. 1a to 1c. It may be noted that the grid size of the first membrane support is significantly smaller than the grid size of the second membrane support. The second membrane support is shown in detail in FIG. 2c. This support is intended in use to support the membrane from the sample container facing side, and this is shown as the left hand side of the membrane provided in FIG. 1a to 1c.

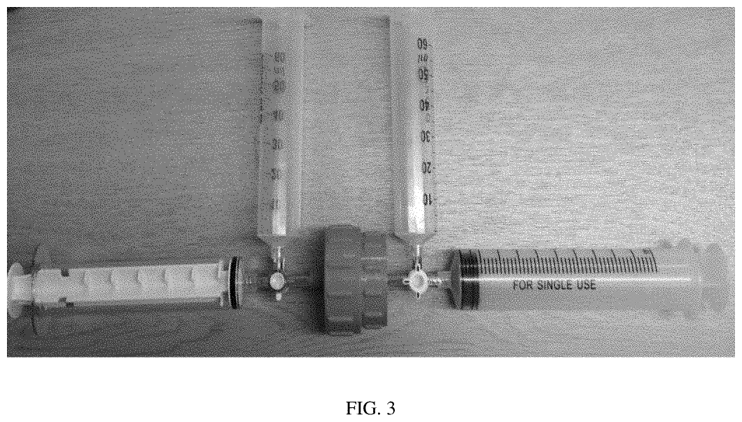

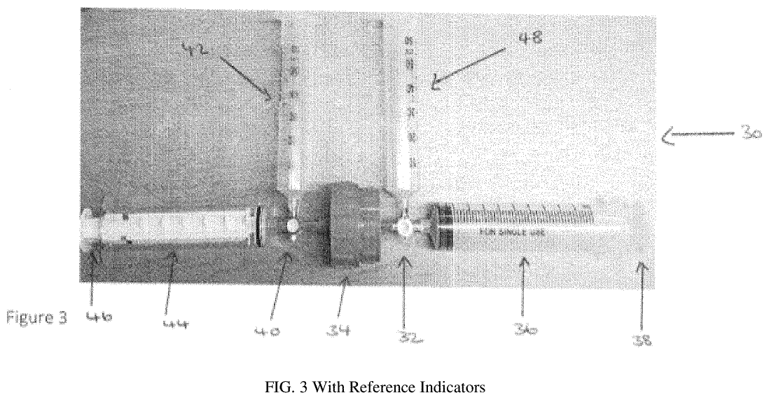

FIG. 3 shows an embodiment 30 of the apparatus of the present invention, in particular suitable for use in the filtration of urine. In use, the fluid connection control mechanism 40 is closed, closing the fluid communication channel between the syringe 42 and the membrane (provided in the membrane holder 34). A sample of urine (approximately 50 ml) is added to syringe 42. The fluid connection control mechanism 32 is closed, closing the fluid communication channel between the membrane (provided in the membrane holder 34) and syringe 36. A partial vacuum is established by extension of the plunger 38 of the syringe 36. The fluid connection control mechanism 32 is opened, opening the fluid communication channel between the membrane and syringe 36, and providing a pressure differential across the membrane. The fluid connection control mechanism 40 is opened, opening the fluid communication channel between the membrane and syringe 36, and the urine sample is pulled or sucked through the membrane due to the pressure differential across the membrane, into syringe 36. As the urine sample is pulled into syringe 36, the partial vacuum is partially relieved, reducing the pressure differential across the membrane. Around 47.5 ml of the urine sample is allowed to flow through the membrane. The remaining urine sample is generally within the membrane housing 34. The fluid connection control mechanism 32 is then closed, closing the fluid communication channel between the membrane and syringe 36 and removing the pressure differential across the membrane.

A check is made to ensure that the fluid connection control mechanisms 32 and 40 are closed, ensuring that the fluid communication channel is closed between the membrane and syringes 44 and 48. Cell preservative fluid is added to syringe 48. A vacuum is established by extension of the plunger 46 of syringe 44. The fluid connection control mechanism 40 is opened, opening the fluid communication channel between the membrane and syringe 44, and providing a pressure differential across the membrane. The fluid connection control mechanism 32 is opened, opening the fluid communication channel between the membrane and syringe 48. The cell preservative fluid is sucked through the membrane due to the pressure differential across the membrane, suspending the cell residue and remaining urine sample which is sucked into syringe 44.

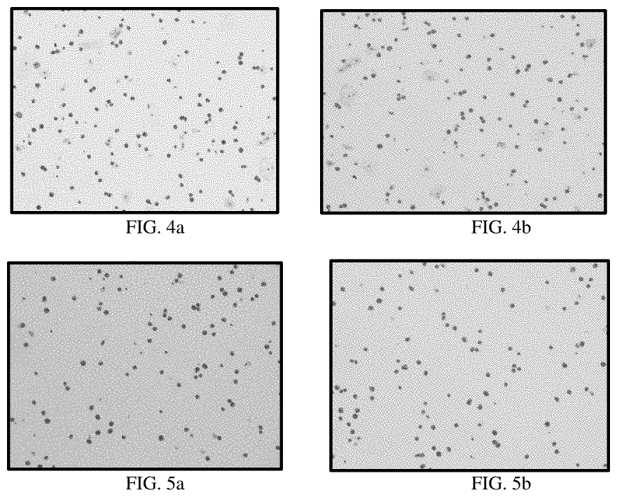

FIGS. 4a and 4b show cell recovery images from a sample which has undergone centrifugation, and FIGS. 5a and 5b show cell recovery images from a sample which has undergone the method of the present invention. The number of cells recovered is broadly equivalent.

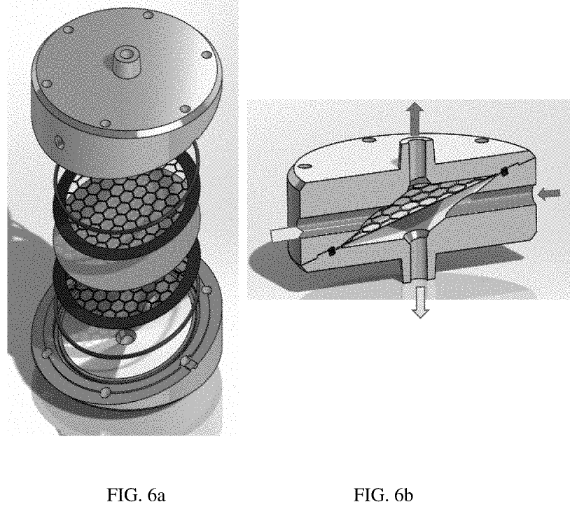

FIGS. 6a and 6b show a further embodiment of the apparatus of the present invention. FIG. 6b shows a membrane housing including four ports.

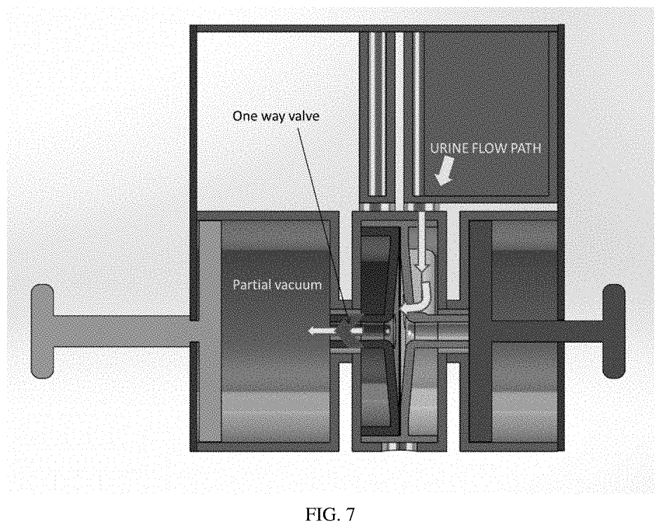

FIG. 7 illustrates an embodiment of the apparatus including a vertically disposed membrane. The fluid sample is pulled through the membrane in a horizontal flow path under the action of a partial vacuum. There is included a one way valve.

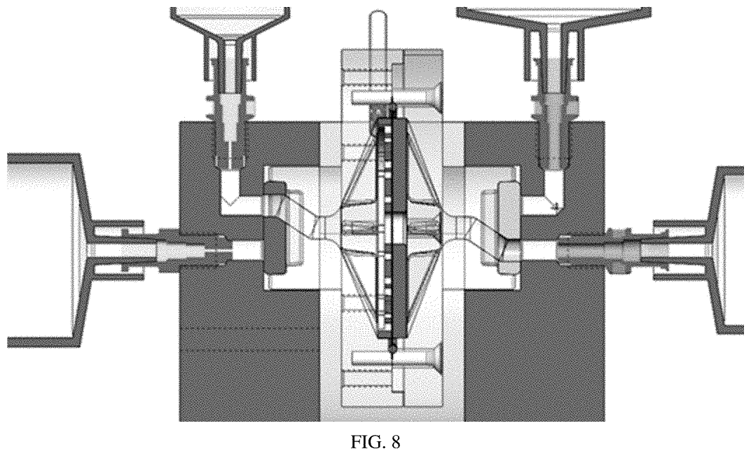

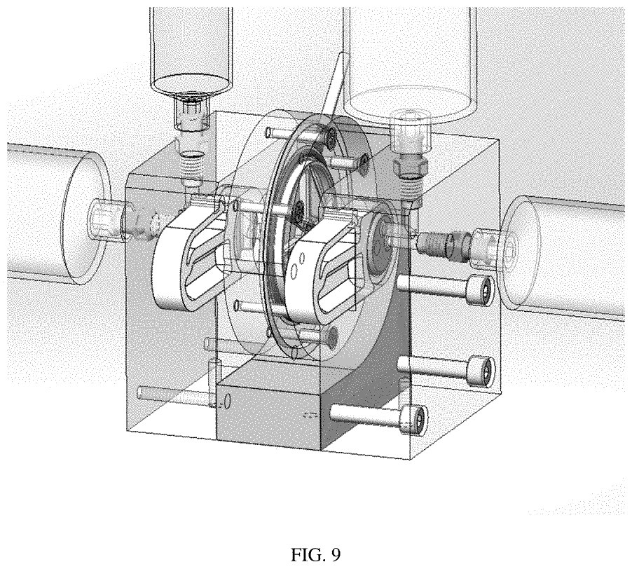

FIG. 8 shows an embodiment the first port is movable between a first position wherein the first port is in fluid communication with the sample container 2, and a second position wherein the first port is in fluid communication with the cell collection container 10; and wherein the second port is movable between a first position wherein the second port is in fluid communication with the filtrate container 8, and a second position wherein the second port is in fluid communication with the backwash fluid container 9. The first and second ports are movable between the first and second positions through rotation of the membrane housing 6. The first and second ports are also movable to a closed position where movement of fluid from the sample container 2 to the membrane is precluded and movement from the membrane to the cell collection container 10 is precluded.

Generally, the apparatus of the present invention includes a flow restriction orifice provided between the second port and the filtrate collection container. The diameter of the flow restriction orifice is around 0.5 mm, the narrowest diameter of the first port is 1 to 2 mm, the narrowest diameter of the second port is 3 mm. The relative diameters of the first and second ports and the flow restriction orifice serve to restrict the flow rate from the membrane to the filtrate container, and reduce the force applied to cells collected on the membrane, thus reducing the risk of damage of the cells. It has been found that where the flow restriction orifice is less than around 0.4 mm, this is associated with some clogging of the membrane.

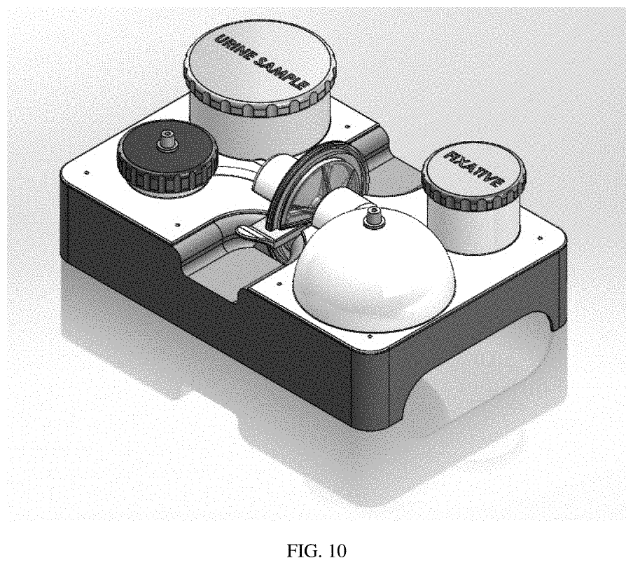

FIG. 10 shows an embodiment of the apparatus of the present invention including a membrane 5 provided vertically. The membrane 5 and membrane housing 6 of this embodiment may be detached and cleaned before reattachment. Alternatively, the membrane and membrane housing may be disposable, and may be detached and replaced.

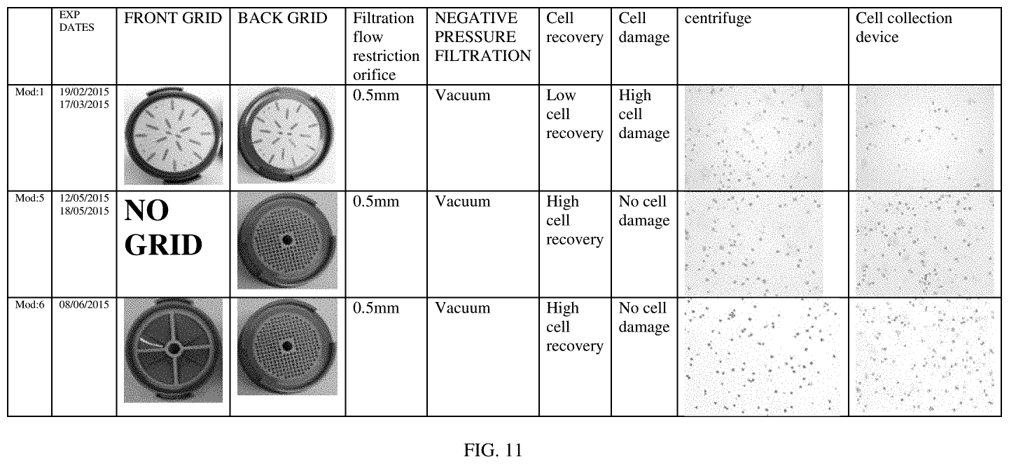

FIG. 11 evidences the effect of different types of membrane support members on the rate of cell recovery and the rate of cell damage. It can be seen that the use of a membrane support member during the filtration process having a relatively low number of relatively large holes is associated with a low cell recovery rate and a low rate of cell damage. In contrast, the use of a grid having a relatively large number of relatively small holes is associated with a far higher cell recovery rate and a low cell damage rate. The results from this embodiment are comparative to, or even better than the rates associated with recovery through the use of a centrifuge.

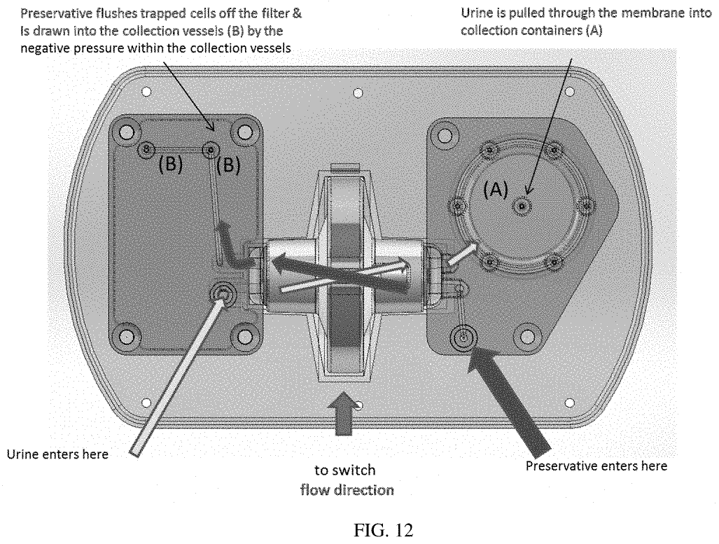

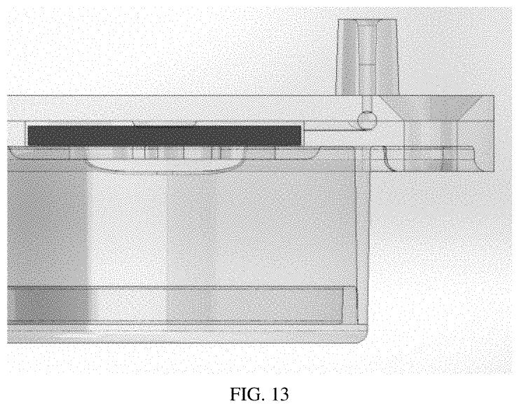

The flow pathways are illustrated in FIG. 12. A urine sample is provided into the sample container via a sample inlet. The urine is pulled through the membrane through the application of a negative pressure across the membrane into the filtrate container (A). Preservative is provided in the backwash fluid container. The preservative is pulled through the membrane by the application of a negative pressure across the membrane, flushing the cells retained on the membrane off the membrane and into the cell collection container (B). The membrane housing may be rotated or slid between a first position wherein the first port is in fluid communication with the sample container, and a second position wherein the first port is in fluid communication with the cell collection container. Such movement of the membrane housing causes the second port to move between a first position wherein the second port is in fluid communication with the filtrate container, and a second position wherein the second port is in fluid communication with the backwash fluid container.

The cell collection container and/or the filtrate container optionally comprise a compartment on the underside (void container) which acts to reduce the negative pressure/create a partial vacuum, generated by the collection vessels. Optionally, there is a one-way valve (red rectangle) which serves to allow air out of the void chamber and seals again when the pressure is equalised which occurs after the preservative (or filtrate) is collected within the collection containers, this serves to prevent preservative (or filtrate) from leaking into the void chamber.

The volume of the void chamber may be modified to increase or reduce the partial vacuum. Typically, the void volume is between 8 ml and 12 ml in the preservative cell collection section and 20-40 ml in the filtrate collection container.

1. An apparatus for collecting or isolating cells from a sample including target cells, the apparatus comprising:
a membrane adapted to retain the target cells thereon housed within a membrane housing,
a first port in fluid communication with the membrane provided towards a first side of the membrane,
a second port in fluid communication with the membrane provided towards a second side of the membrane,
a sample container having at least one wall defining an interior space suitable to house the sample, provided on the first side of the membrane and configured to allow fluid flow from the first side of the membrane to the second side of the membrane,
a cell collection container provided towards the first side of the membrane,
a filtrate container provided towards the second side of the membrane,
a backwash fluid container provided towards the second side of the membrane and configured to allow fluid flow from the second side of the membrane to the first side of the membrane,
wherein the first port is movable between a first position wherein the first port is in fluid communication with the sample container, and a second position wherein the first port is in fluid communication with the cell collection container;
wherein the second port is movable between a first position wherein the second port is in fluid communication with the filtrate container, and a second position wherein the second port is in fluid communication with the backwash fluid container;
wherein the first port is movable between the first position and the second position through rotation of the membrane housing, and the second port is movable between the first position and the second position through rotation of the membrane housing; and
wherein the orientation of the membrane to the containers remains constant during movement of the first port between the first position and the second position, and during movement of the second port between the first position and the second position.

2. (canceled)

3. The apparatus as claimed in claim 1, wherein the first port comprises or consists of an aperture in the membrane housing, and the second port comprises or consists of an aperture in the membrane housing, and wherein the sample container, the cell collection container, the filtrate container and the backwash fluid container are provided externally of the membrane housing.

4. The apparatus as claimed in claim 3, wherein the first and second ports each comprise an aperture within the membrane housing and a channel leading from the inside wall of the membrane housing towards the membrane.

5-6. (canceled)

7. The apparatus of claim 1, wherein the first port is in the first position when the second port is in the first position and a fluid pathway is provided from the sample container, through the membrane, to the filtrate container, and
wherein the first port is in the second position when the second port is in the second position and a fluid pathway is provided from the backwash fluid container, through the membrane, to the cell collection container.

8. (canceled)

9. The apparatus of claim 1, wherein the first port provides fluid communication to either the sample container or the cell collection container, and the second port provides fluid communication to either the filtrate container or the backwash fluid container.

10. The apparatus as claimed in claim 9, wherein the membrane housing precludes fluid access between the membrane and the cell collection container where the first port is in the first position, and the membrane housing precludes fluid access between the membrane and the sample container where the first port is in the second position, and
wherein the membrane housing precludes fluid access between the membrane and the backwash fluid container where the second port is in the first position, and the membrane housing precludes fluid access between the membrane and the filtrate container where the second port is in the second position.

11. (canceled)

12. The apparatus as claimed in claim 1, wherein the first port is movable between the first position, the second position and a closed position, and in the closed position, movement of fluid from the sample container to the membrane is precluded and movement from the membrane to the cell collection container is precluded,
wherein the second port is movable between the first position, the second position and a closed position, and
wherein in the closed position movement of fluid from the membrane to the filtrate container is precluded and movement from the backwash fluid container to the membrane is precluded.

13-14. (canceled)

15. The apparatus as claimed in claim 1, wherein the ports are adapted to be non-removable during the use of the apparatus in a filtration process.

16. The apparatus as claimed in claim 1, further comprising more than two ports in fluid communication with the membrane.

17. The apparatus as claimed in claim 1, wherein the membrane is disposed non-horizontally and the fluid flow path of the sample through the membrane is non-vertical, and the apparatus is adapted to apply a pressure differential across the membrane.

18. The apparatus of claim 1, wherein the membrane is disposed substantially horizontally and the fluid flow path of the sample through the membrane is substantially vertical.

19. The apparatus as claimed in claim 1, comprising a flow restriction orifice between the second port and the filtrate container wherein the narrowest width or diameter of the first port is as least two times greater than the narrowest width or diameter of the flow restriction orifice.

20. The apparatus as claimed in claim 1, wherein the first side of the membrane is supported by a first grid or mesh membrane support, and the second side of the membrane is supported by a second grid or mesh membrane support and the grid or mesh size of the first membrane support is at least 5 times greater than the grid or mesh size of the second membrane support.

21. The apparatus as claimed in claim 1, wherein the sample container comprises a mechanism to close the passage between the sample container and the membrane once the sample container has emptied of the sample.

22. The apparatus as claimed in claim 21, wherein the mechanism comprises a floating ball shut-off valve.

23-24. (canceled)

25. A method for collecting cells from a fluid sample including the target cells, the method comprising:
providing the apparatus as claimed in claim 1;
applying an initial pressure differential across the membrane;
allowing the sample to begin to pass through the membrane;
applying a secondary pressure differential across the membrane opposite to the initial pressure differential;
allowing a backwash fluid to pass through the membrane in a second direction, opposite to the first direction; and,
collecting the cells retained on the membrane.

26-34. (canceled)

35. A method of screening biological samples to identify those associated with an increased risk of a disease including:
a) collecting cells from a sample in accordance with the method of claim 25,
b) removing the collected cells from the membrane;
c) measuring the presence, and/or concentration of one or more target cells in the collected cell sample of step b; and
d) analysing the measurements of step c for evidence of an increased risk of a disease state.

36. (canceled)

37. A method of diagnosing the risk of a patient having a disease or is likely to have the disease in the future including:
collecting cells from a sample in accordance with the method of claim 25,
measuring one or more levels of the target cells in the sample,
calculating a risk score for the patient, and
using the risk score to identify a likelihood that the patient has the disease or is likely to have the disease in the future.

38. (canceled)

39. A system for performing a method of screening biological samples using an apparatus and a suitably programmed computer:
wherein the apparatus comprises:
a membrane adapted to retain the target cells thereon,
a first port in fluid communication with the membrane provided towards a first side of the membrane,
a second port in fluid communication with the membrane provided towards a second side of the membrane,
a sample container having at least one wall defining an interior space suitable to house the sample, provided on the first side of the membrane,
a cell collection container provided towards the first side of the membrane,
a filtrate container provided towards the second side of the membrane,
a backwash fluid container provided towards the second side of the membrane,
wherein the first port is movable between a first position wherein the first port is in fluid communication with the sample container, and a second position wherein the first port is in fluid communication with the cell collection container; and
wherein the second port is movable between a first position wherein the second port is in fluid communication with the filtrate container, and a second position wherein the second port is in fluid communication with the backwash fluid container; and,
the computer is programmed for carrying out or assisting in one or more of measuring the levels of the target cells in the sample; calculating a risk score; using the risk score to identify a likelihood that the patient suffers from a disease or has an increased likelihood of suffering from a disease in future; and displaying information related to the likelihood such as the measured target cell levels, the risk score, the likelihood that the patient suffers from a disease, or has an increased likelihood of suffering from a disease in future and a reference risk score; and,
the method comprises:
providing the apparatus;
applying an initial pressure differential across the membrane;
allowing the sample to begin to pass through the membrane;
applying a secondary pressure differential across the membrane opposite to the initial pressure differential;
allowing a backwash fluid to pass through the membrane in a second direction, opposite to the first direction; and,
collecting the cells retained on the membrane removing the collected cells from the membrane;
measuring the presence, and/or concentration of one or more target cells in the collected cell sample of step b; and,
analysing the measurements of step c for evidence of an increased risk of a disease state.

40. (canceled)

41. The apparatus as claimed in claim 1, wherein the sample container and the backwash fluid container are provided in an upright position in use.
